(19) United States
(12) Patent Application Publication
(10) Pub. No.: US 2022/0313865 A1
(43) Pub. Date: Oct. 6, 2022

(54) HALO Sanitizer Respirator

(71) Applicant: Lawrence Sadler, Palmetto, FL (US)

(72) Inventor: Lawrence Sadler, Palmetto, FL (US)

(21) Appl. No.: 17/711,956

(22) Filed: Apr. 1, 2022

Related U.S. Application Data
(60) Provisional application No. 63/169,408, filed on Apr. 1, 2021.

(51) Int. Cl. A61L 9/20; A62B 18/02; A41D 13/11

(52) U.S. Cl. CPC A61L 9/20; A62B 18/025; A41D 13/11; A61L 2209/12

(57) ABSTRACT

One embodiment of The Halo Sanitizer Respirator system is an electronic facial respirator featuring a shielded halo chamber to irradiate pathogens with a 1″ diameter halo ring of chip-on-board package UV 265 nm LEDs. The LEDs face each other at an overall 180° inward-facing angle. The halo ring is positioned around the inner circumference of a shielded mirrored chamber. The halo ring concentrates and amplifies UVA, UVB and UVC rays. The halo respirator also features protective, supportive, flow-directing baffle vane panels positioned on the intake and the outflow points of the shielded mirror chamber to direct airflow, support chamber components, and to prevent radiation from escaping. The sanitizing respirator is further equipped with a rechargeable, detachable battery power unit, designed to be recharged in a separate, sanitizing/charging dock.

### CROSS-REFERENCES TO RELATED APPLICATIONS

This application claims the benefit of U.S. Provisional Patent Application No. 63/169,408 entitled “HALO Sanitizer Respirator” and filed on Apr. 1, 2021 for Lawrence Sadler, which is incorporated herein by reference.

### FIELD

This disclosure relates generally to methods and devices for harnessing and directing ultraviolet (“UV”) light in a respirator to kill pathogens, as well as the field of electrically-charged respirators.

### BACKGROUND

Current research indicates that irradiation of pathogens such as infectious virus particles of severe acute respiratory syndrome coronavirus 2 (“SARS-CoV-2”) with UV radiation for periods as low as 0.08 seconds may reduce viral titer populations by more than 95%. Short-wavelength UV light between 255 and 280 nm (e.g. ultraviolet-C radiation (“UVC)) effectively disinfects viruses, including SARS-CoV-2, because wavelengths in this range are readily absorbed by nucleic acids, causing damage to the deoxyribonucleic acid (“DNA”) and ribonucleic acid (“RNA”) of microorganisms.

Since viral pathogens are significantly reduced by 265-nm UV irradiation, several UV-light emitting devices for the inactivation of SARS-CoV-2 at various sites have been developed. However, no stable device has heretofore effectively incorporated UV radiation into a sanitizing respirator mask that can consistently irradiate pathogens, focusing and augmenting the radiation by shape and design, powered via rechargeable battery with a separate charging & sanitizing dock chamber, while preventing additional pathogens to enter the sides of the respirator, all while preventing desired-radiation to escape.

What is needed is an effective, portable, stable, sanitary rechargeable way to incorporate 265 nm UVC irradiation technology in a sanitizing facial respirator.

### SUMMARY

One embodiment of The Halo Sanitizer Respirator system is an electronic facial respirator featuring a shielded halo chamber to irradiate pathogens with a 1″ diameter halo ring of chip-on-board package UV 265 nm light emitting diodes (“LEDs”). The LEDs face each other at a 180° inward-facing angle. The halo ring is positioned around the inner circumference of a shielded mirrored chamber. The halo ring concentrates and amplifies ultraviolet A-rays (“UVA”), ultraviolet B-rays (“UVB”) and UVC rays.

The halo respirator also features protective, supportive, flow-directing baffles (baffle vanes or panels) positioned on the intake and the outflow points of the shielded mirror chamber to direct airflow, support chamber components, and to prevent radiation from escaping.

The sanitizing respirator is further equipped with a rechargeable, detachable battery power unit, designed to be optionally recharged in a separate charging & sanitizing dock chamber.

The described features, structures, advantages, and/or characteristics of the

N subject matter of the present disclosure may be combined in any suitable manner in one or more examples and/or implementations. In the following description, numerous specific details are provided to impart a thorough understanding of examples of the subject matter of the present disclosure. One skilled in the relevant art will recognize that the subject matter of the present disclosure may be practiced without one or more of the specific features, details, components, materials, and/or methods of a particular example or implementation. In other instances, additional features and advantages may be recognized in certain examples and/or implementations that may not be present in all examples or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

### BRIEF DESCRIPTION OF THE DRAWINGS

In order that the advantages of the subject matter may be more readily understood, a more particular description of the subject matter briefly described above will be rendered by reference to specific examples that are illustrated in the appended drawings. Understanding that these drawings, which are not necessarily drawn to scale, depict only certain examples of the subject matter and are not therefore to be considered to be limiting of its scope, the subject matter will be described and explained with additional specificity and detail through the use of the drawings, in which:

FIGS. 1A and 1B illustrate front views of separate embodiments of the technology according to one or more examples of the present disclosure.

FIG. 2 illustrates a side cross-sectional view of the of the technology according to one or more examples of the present disclosure.

FIG. 3 illustrates an exploded view of the of the technology according to one or more examples of the present disclosure.

FIG. 4 illustrates a side cross-sectional view of the of the technology according to one or more examples of the present disclosure.

FIGS. 5A and 5B illustrate side views of the technology according to one or more examples of the present disclosure.

FIG. 6 illustrates a perspective elevational view of an optional separate charging dock station & sanitizing dock chamber embodiment according to one or more examples of the present disclosure.

### DETAILED DESCRIPTION

Reference throughout this specification to “one example,” “an example,” or similar language means that a particular feature, structure, or characteristic described in connection with the example is included in at least one example of the present disclosure. Appearances of the phrases “in one example,” “in an example,” and similar language throughout this specification may, but do not necessarily, all refer to the same example. Similarly, the use of the term “implementation” means an implementation having a particular feature, structure, or characteristic described in connection with one or more examples of the present disclosure, however, absent an express correlation to indicate otherwise, an implementation may be associated with one or more examples.

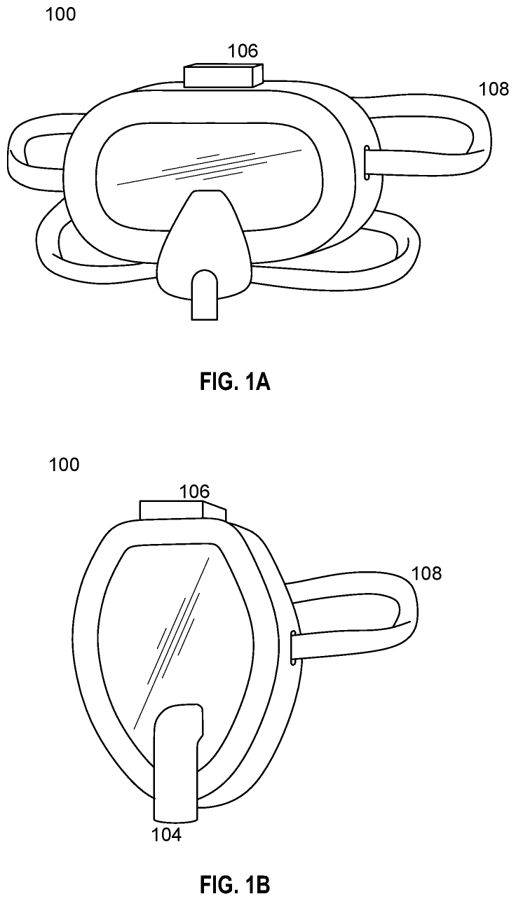

FIG. 1A illustrates a “Halo” Sanitizer Respirator 100 system as a non-disposable electronic respirator. The respirator comes in multiple form factors, two of which are shown as FIGS. 1A and 1B.

The first form factor (FIG. 1A) covers the nose and mouth (and optionally includes separate eye protection), whereas the second form factor (FIG. 1B) covers the entire face. Both embodiments shown feature adjustable straps 108 and rechargeable battery power unit 106. This technology is electronic, therein requiring no filter replacements.

The technology utilizes 265 nm UVA, UVB, and UVC technology within a shielded halo mirror chamber 104 to create a non-filtered free-flowing design providing a sanitizing protection shield for the user. The halo respirator 100 kills the germs and viruses on contact through intense UVC radiation while eliminating the pathogens and mold from entering the respiratory system. The unit therefore sanitizes both the inhalation and the exhaust of the user, allowing the user to breathe naturally while preventing the spread of infectious diseases.

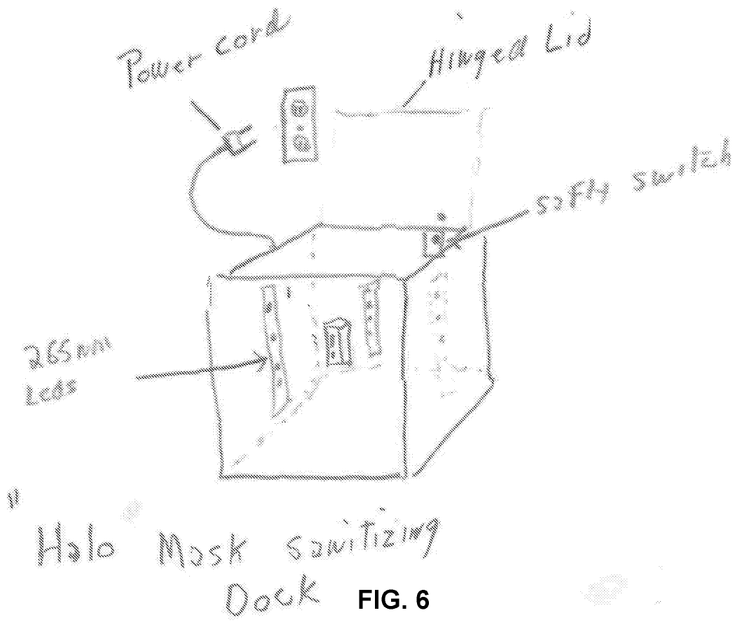

The sanitizing respirator is further equipped with a rechargeable battery power unit 106 which can be charged with its own separate sanitizing/charging dock and sanitization chamber (shown in FIG. 6).

The Halo Respirator is profoundly effective in preventing toxic biologic aerosols, including infectious agents such as viruses and bacteria, from entering the eyes, nose, and mouth, and will not degrade like most cartridge filtered systems over the duration of use because of its electronic nature. This non-disposable full-face respirator 100 “Halo” system is designed for months of continuous operation unlike disposable single use systems. This technology is totally safe. The “Halo” respirator is built for comfort with a soft surface face seal, and is fully adjustable via, inter alia, adjustable straps 108.

The “Halo” respirator 100 is also equipped with a red LED indicator light to display continuous operation, with a runtime of more than 12 hours. With its free-flowing design, the “Halo” respirator 100 is built for easy maintenance, for long term use, and with the built-in easy-care air sanitizing system the unit eliminates the need for cartridge and paper filter replacements making the unit self-contained and very cost efficient.

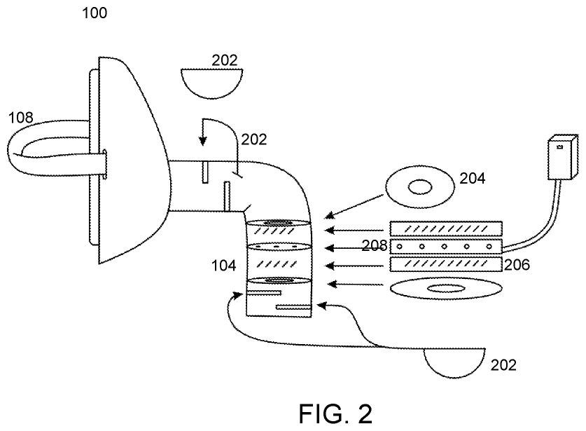

Referring to FIG. 2, the instant technology is shown as a cross sectional side view featuring components of the respirator 100 chamber area 104. The chamber area 104 features a plurality of interspersed baffles 102 positioned in the interior area of the chamber. These protective, supportive, airflow-directing baffles 202 (e.g. vanes/panels) are positioned throughout the inhale/exhaust points of the shielded mirror chamber 104. The baffles 202 direct airflow, support chamber 104 components, and prevent radiation from escaping. Using similar fluid mechanics principles to that of bulkheads, the interspersed parallel positioning of the baffles 202 also aids pathogen-killing efficacy and component support.

The chamber 104 comprises mirror discs 204 and mirror strips 206, further focusing and augmenting the instant radiation. The chamber's 104 inner surface is further lined with additional material that reflect ultraviolet radiation. The operative LED halo ring 208 is herein shown connected to a 9-volt battery pack 106.

In one embodiment, the halo ring 208 may be a 1″ diameter halo ring 208 of chip-on-board package UV 265 nm LEDs 210. The LEDs may face each other at an overall 180° inward-facing angle (full circumference 360° if the halo ring 208 is lining the full circumference of the chamber 104).

In one embodiment, the halo ring 208 is positioned around the inner circumference of a shielded mirrored chamber 104. The halo ring 208 concentrates and amplifies UVA, UVB and UVC rays.

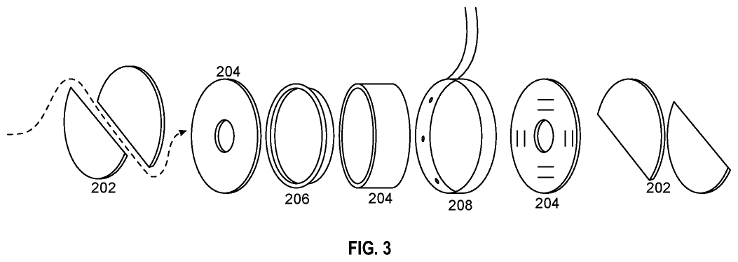

Referring to FIG. 3, the chamber 104 technology may comprise protective baffles 202, as well as front face and main-central mirrored shield discs 204, as well as coated mirror strips 206 lining the interior circumference of the chamber 104 in the form of mirror rings 206. [The mirrored strip 206 lining the chamber 104 will, so positioned, form the ring shape].

FIG. 3 further shows the central LED 210 Strip 208 lining the chamber's circumference to form an LED ring 208. In the instant embodiment, the ring 208 comprises a COB [chip on board package] of a plurality of 265 nm LEDs 210). While the instant FIG. 3 shows six LEDs affixed to the strip/ring 208, industry-standard 9 LEDs 210 may be used, or more.

In the embodiment shown, the mirrored face shields 204 are herein mirrored discs 204. In the embodiment shown, the mirror strips 206 are Titanium dioxide (“TiO2”) coated rings, which further augment the LEDs' 210 light.

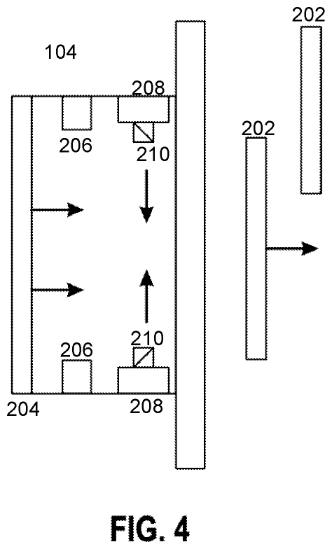

FIG. 4 illustrates a chamber 104 embodiment with a 1″ diameter “halo” ring 208 of chip-on-board UV 265 nm LEDs 210 (as a cob package in the form of a halo ring 208). The instant positioning ensures the LEDs 210 face each other at an 180° inward-facing angle (or 360° angle if the complete 360° inner circumference were shown). The halo ring 208 is positioned around the inner circumference of the shielded mirrored chamber 104 (said chamber optionally being a vacuum chamber). The halo ring 208 concentrates and amplifies the UVA, UVB and the UVC rays.

The halo respirator 100 also features protective, supportive, flow-directing baffle 202 vane panels positioned on the intake and the outflow points inside and outside the chamber 104 to direct aerosols, secure respirator components' positions, and to prevent radiation from escaping.

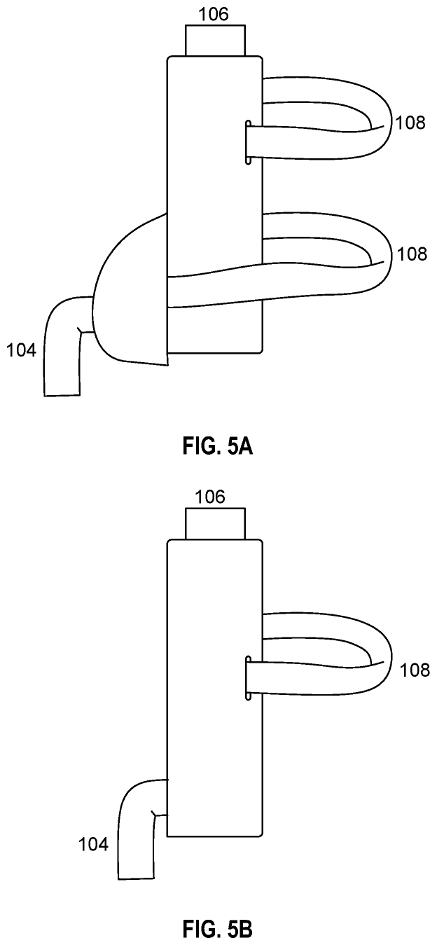

FIGS. 5A and 5B illustrate side views of the respirator 100, the instant embodiments featuring adjustable face-holding straps 108 and rechargeable battery power unit 106.

FIG. 6 illustrates the technology's optional separate charging dock station & sanitizing dock chamber. This unit comprises, inter alia, their own strips of sanitizing 265 nm LEDs 210, optional power cord, hinged lid, Fly Switch. This unit, and various embodiments thereof, recharge the technology's battery unit 106, while also providing additional sanitation for items placed inside the chamber itself via multiple wavelengths of UV radiation disclosed in the instant technology.

In the above description, certain terms may be used such as “up,” “down,” “upper,” “lower,” “horizontal,” “vertical,” “left,” “right,” “over,” “under” and the like. These terms are used, where applicable, to provide some clarity of description when dealing with relative relationships. But these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.” Moreover, unless otherwise noted, as defined herein a plurality of particular features does not necessarily mean every particular feature of an entire set or class of the particular features.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The schematic flow chart diagrams included herein are generally set forth as logical flow chart diagrams. As such, the depicted order and labeled steps are indicative of one example of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present subject matter may be embodied in other specific forms without departing from its spirit or essential characteristics. The described examples are to be considered in all respects only as illustrative and not restrictive. All changes which come within the meaning and range of equivalency of the claims are to be embraced within their scope.

What is claimed is:

1. An electrically-powered sanitizing respirator comprising a respirator mask with a mask body, a breathing chamber with an interior space, and at least one ultraviolet (“UV”)-emitting light source to irradiate air flowing through the chamber.

2. The respirator of claim 1 wherein the chamber interior has an inner surface lined with material that reflects ultraviolet radiation.

3. The respirator of claim 1 wherein the UV-emitting light source is a strip comprising a plurality of chip-on-board UV 265 nm light emitting diodes (“LEDs”).

4. The respirator of claim 1 wherein the electrical power is provided by a detachable, rechargeable battery power unit that may be recharged in a separate recharging dock station chamber that comprises its own sanitizing UV-emitting light source.
